(19) United States
(12) Patent Application Publication
(10) Pub. No.: US 2017/0119470 A1
(43) Pub. Date: May 4, 2017

(54) DEVICE AND METHOD FOR FRAGMENTING ORGANO-MINERAL CONCRETIONS

(71) Applicant: LITHOTECH MEDICAL LTD., Katzin (IL)

(72) Inventors: Valery DIAMANT, Katzrin (IL); Gennady CHEPOVETSKY, Halutz (IL); Sergey EREMENKO, Sevastopol (RU); Alexey EREMENKO, Krasnodar (RU); Alexey MARTOV, Moscow (RU); Alexander GUDKOV, Tomsk (RU); Marat LERNER, Tomsk (RU); Vladimir CHERNENKO, Tomsk (RU)

(21) Appl. No.: 15/318,440

(22) PCT Filed: Sep. 2, 2015
(86) PCT No.: PCT/IL2015/050878

§ 371 Date: Dec. 13, 2016

Related U.S. Application Data
(60) Provisional application No. 62/044,273, filed on Aug. 31, 2014.

(51) Int. Cl. A61B 18/26; A61B 17/22

(52) U.S. Cl. CPC A61B 18/26; A61B 17/22022; A61B 2017/22011; A61B 2018/00994

(57) ABSTRACT

A medical device and method for breaking a concretion in a body into smaller pieces are described. The device comprises a combined probe including a laser waveguide probe and a nanosecond electro-pulse lithotripter probe. The method includes applying the laser waveguide probe to the surface of the concretion, and treating the surface with laser radiation to create a defect. The method also includes applying the nanosecond electro-pulse lithotripter probe to the area of the defect created by the laser waveguide probe, to provide a spark electrical discharge through the concretion.

### FIELD OF THE INVENTION

This invention relates to a device and method for fragmenting abnormal concretions in body cavities, and in particular, to a medical technique for fragmenting hard solid calculous formations in the ducts and cavities of a living body.

### BACKGROUND OF THE INVENTION

Due to numerous reasons, all manner of organo-mineral concretions can be formed in the cavities of organs of humans and other mammals Occlusions of various blood vessels, including calcified vessels, salivary gland calculi, urinary system calculi, biliary calculi, and so forth are examples of such concretions. Disintegration of such concretions is a highly important issue today.

Several techniques are employed in clinical practice for breaking abnormal concretions appearing in the biliary and/or urinary system of a human body into pieces for further removal of the pieces from the body. To cure the most complicated forms of nephroureterolithiasis (large, multiple, and coralloid renal calculi, “impacted” and large ureteral calculi, etc.), endourological methods are increasingly used. In particular, the use of percutaneous and transurethral contact lithotripsy makes it possible to reduce perioperative risks of remote lithotripsy and open lithotomy, as well as to reduce the duration of inpatient and outpatient treatment.

Although procedures have varied, most of them have involved dilatating, anesthetizing and lubricating the urinary or biliary tract, and then attempting to grasp the calculus for crushing it, and then dragging it out.

The principal methods in current use for intracorporeal lithotripsy are the ultrasonic, pneumatic, electrokinetic, laser, and electrohydraulic methods. For example, intra-corporeal shock wave lithotripsy employs high-energy shock waves to fragment and disintegrate calculi. Ultrasonic lithotripsy technique is known that utilizes an ultrasound probe emitting high-frequency ultrasonic energy towards a concretion. For this technique, direct contact of the probe tip and stone is essential for effectiveness of ultrasonic lithotripsy.

Each method has advantages and drawbacks. For instance, in applying ultrasonic lithotripsy, only rigid probes and rigid endoscopes can be used, and the scope of its application is currently limited primarily to renal calculi. Impact lithotripsy (pneumatic or electrokinetic methods) is one of the most effective and safe methods for contact fragmentation of stones. Use of such lithotripters is also restricted to rigid endoscopes, and retrograde calculus propulsion during transurethral ureterolithotripsy is regarded as a drawback of this method. The electrohydraulic lithotripsy (EHL) and laser lithotripsy methods, being effective methods for contact crushing, can be used with both rigid and flexible endoscopes, which substantially expand the scope of their application in modern urology.

The EHL technique is effective in breaking urinary stones into pieces small enough for basket extraction or simple passage. When EHL is selected to affect destruction of the stone, the EHL probe is placed in proximity to the stone. By means of an electrical discharge, a shock wave is produced which impacts the surface of the stone and produces tiny cracks. When enough cracks have been made, the stone shatters into small pieces. The individual pieces can then be attacked one at a time, or they can further be removed by basket extraction. However, the electrohydraulic method gives rise to a much greater number of complications as compared to other methods, since the produced shock wave damages the surrounding tissues when the discharge occurs too close to the urinary tract walls.

On the other hand, laser based technologies are widely used today in medicine for various purposes, including fragmentation various types of organo-mineral concretions. Lasers are known as an alternative source of energy in lithotripsy, especially for the destruction of renal stones. Various types of laser lithotripsy probes with a variety of laser sources, including pulsed dye laser, alexandrite laser, neodymium laser, holmium laser and other lasers, have been developed. Laser fragmentation is safer than EHL, but it takes longer for fragmentation to occur, and laser lithotripsy requires more costly equipment. Furthermore, frequent damage to the flexible ureteropyeloscope, which occurs due to breakage of the laser fiber inside the bent endoscope, is a major drawback of this technique.

A lithotripsy technique of nanosecond electro-pulse destruction of materials is also known in the art (see, for example, U.S. Pat. No. 7,087,061 and U.S. Pat. Publ. No. US 2007/0021754) that can be used for safe contact fragmentation of calculi in all parts of a patient's urinary tracts. This technique employs probes of various diameters, compatible with both rigid and flexible endoscopes.

The nanosecond electric breakdown carried out through the calculus results in the rise of a plasma channel, occurrence of micro-explosions, and the emergence of numerous thermo-mechanical tensile stresses in the concretions. However, due to its operating principle, the nanosecond electro-pulse lithotripsy (NPL) is essentially different from the electrohydraulic lithotripsy (EHL). Contrary to electrohydraulic destruction, employing electrodes which are not in direct contact with the object, electro-pulse destruction utilizes a probe with electrodes which are placed directly on the object's surface, and uses short, nanosecond electric pulses with steep fronts to fragment stones. This technique is based on the Vorob'evs effect that provides certain features of the discharge observed when a solid dielectric in contact with two rodlike electrodes is placed in a liquid dielectric medium, and a voltage pulse with increasing front is applied to the electrodes. According to this effect, when the pulse front slope is not so steep (e.g., the pulse rise time is more than about 0.3 microseconds), a discharge develops in the surrounding liquid over the solid dielectric surface, rather than penetrating into the solid body. On the other hand, in the case of a sufficiently steep slope of the pulse front (e.g., the pulse rise time is less than about 100 ns), the discharge current propagates through the solid. This gives rise to tensile mechanical effects in the calculus, which causes its cracking and, finally, fragmentation (see, for example, G. A. Masyats, Technical Physics Letters, Vol. 31, No. 12, 2005, pp. 1061-1064. Translated from Russian from Pis'ma v Zhurnal Tekhnicheskoi Fiziki, Vol. 31, No. 24, 2005, pp. 51-59).

Shock waves during application of electrohydraulic lithotripsy (EHL) technique may result in serious damage to surrounding tissues and for this reason the EHL technique virtually ceased being in use for endoscopic fragmentation. On the other hand, when nanosecond electro-pulse lithotripsy (NPL) technique is used for fragmentation of calculi or other concretions, the energy of the electric pulse is released directly within the bulk of the solid body being fragmented, rather than in the surrounding liquid. Thus, the NPL technique requires considerably less energy for disintegration of concretions than the EHL technique, and makes the method safer.

Lasers used in medicine offer a wide range of wavelengths from the ultraviolet band to the near infrared band, including the visible part of the spectrum. Primary lasers currently in use are the Holmium, Ho:YAG laser with a wavelength of 2.140 μm, the Neodym, Nd:YAG laser with a wavelength of 1.064 μm, the Kalium titanyl phosphate KTP:Nd:YAG (SHG) laser with a wavelength of 0.532 μm, the Lithium borat LBO:Nd:YAG (SHG) laser with a wavelength of 0.532 μm, the Thulium Tm:YAG laser with a wavelength of 2.013 μm, Dye Lasers with wavelengths in a wide range, CO—lasers with a wavelength of 10.6 μm, and Diode lasers with wavelengths of 0.830 μm, 0.940 μm, 0.980 μm, 1.318 μm, and 1.470 μm.

Laser methods for fragmentation of organo-mineral concretions are well-known and are widely used nowadays. An example of such use can be the Ho: YAG laser with a wavelength of 2.1 μm, employed in urology for the fragmentation of urological stones. The interaction of laser radiation with the surface of tissues and concretions, or the fragmentation of organo-mineral concretions by using lasers is related to a combination of three principal mechanisms: photothermal, photomechanical, and the cavitation bubble effect, that is to say, a shockwave mechanism. The photothermal and photomechanical effects are related to the direct absorption of laser energy by the body being irradiated. Molecules of liquid contained in a body, be it one composed of biological tissues or organo-mineral concretions, absorb a certain wavelength of laser light, which results in their evaporation. Moreover, the high temperature caused by the energy of the laser pulse has the effect of destabilizing the chemical structure of organo-mineral concretions, such that the laser creates a crater on the surface of the body being irradiated. In addition, the laser beam forms spherical cavitation bubbles, which then produce a shock wave as a result of their collapse. This shock wave produces a photoacoustical effect that also promotes fragmentation of organo-mineral concretions. Therefore, a phenomenon may here be observed which is similar to that observed during electrohydraulic lithotripsy. In cases where laser radiation directly affects biological tissues, there occurs a thermic effect whose depth depends on the laser wavelength, which is related to the laser's coefficient of absorption by water, tissues, etc.

Investigations of the Applicant have demonstrated that NPL is the most effective method for destroying biological concretions as compared to the laser or electrohydraulic method.

However, the effectiveness of this method depends fundamentally on the physical properties of the concretion, such as its density and structure. As the density of a concretion rises, the effectiveness of NPL is reduced (see for example, Alexey Martov, Valery Diamant, Artem Borisik, Andrey Andronov, and Vladimir Chernenko. Comparative in Vitro Study of the Effectiveness of Nanosecond Electrical Pulse and Laser Lithotripters. JOURNAL OF ENDOUROLOGY, 2013, V. 27, N. 10, P. 1287-1296; Alexey Martov, Alexander Gudkov, Valery Diamant, Gennady Chepovetsky, and Marat Lerner. Investigation of Differences between Nanosecond Electro-pulse and Electrohydraulic Methods of Lithotripsy: A Comparative In Vitro Study of Efficacy. JOURNAL OF ENDOUROLOGY, 2014, V. 28, N. 4, P. 437-445.

### SUMMARY OF THE INVENTION

As described above, laser techniques allow practically any calculus to be fragmented, but the efficiency of such a laser lithotripter is not high, because a considerable amount of time is required to fragment even a small stone.

On the other hand, nanosecond electro-pulse lithotripsy (NPL) is a highly effective and relatively safe technique that allows for contact fragmentation of organo-mineral concretions in all parts of the urinary tract, as well as in other hollow cavities of organs, such as blood vessels, biliary ducts, etc. Investigation of the Applicants revealed that this technique is much more effective for the fragmentation of concretions than other methods in use today, such as the laser and EHL techniques. However, the efficacy of NPL, as well as some other methods of contact lithotripsy, depends on the density and structure of organo-mineral concretions. Thus, there is dependence between the properties of organo-mineral concretions and the cumulative energy required for their fragmentation.

At the same time, when the surface of organo-mineral concretions is irradiated by laser radiation of some wavelengths, such radiation may change the surface properties of the organo-mineral concretions. It was found by the Applicants that even if this laser radiation has low energy, being insufficient to fragment concretions, the defects which can be created in the concretions may essentially affect the efficacy of NPL, if this technique is used after laser irradiation.

Thus, there is a need in the art for, and it would be useful to have a novel medical device and method for intracorporeal lithotripsy that provide fast and easy fragmentation of any organo-mineral concretions and their pieces inside hollow cavities of organs. In addition, it would be advantageous to reduce the procedure time and lessen the likelihood of injury to adjacent body tissues during treatment.

The term “concretion” as used herein refers to solid calculous formations of urates, oxalates and phosphates, e.g. gallstones, kidney stones, cystine stones and other calculi, lodged in the ducts and cavities of a living body.

It would be advantageous to combine the effects of laser lithotripsy and NPL in a device which is capable to be safely introduced into the confined space of the individual ureter, urinary bladder or biliary tract, blood vessels etc. and create cracks and/or shatter the concretion into smaller pieces.

It would be useful to have a method that can provide fragmentation of organo-mineral concretions due to applying NPL together with the preliminary effect of laser radiation on the concretion. The specified method can open up new horizons for fast fragmentation of large and dense organo-mineral concretions, for example, large dense stones or staghorn stones in the urinary tract, when used in clinical practice using retrograde access.

The present invention satisfies the aforementioned need by providing a novel medical device for breaking a concretion in a body into smaller pieces that allows a user to exert the combined effect of laser radiation and NPL onto organo-mineral concretions, so they are fragmented more rapidly and efficiently, when compared with the use of laser radiation and NPL separately.

The medical device includes a combined probe including a laser waveguide probe and a nanosecond electro-pulse lithotripter probe.

The medical device also includes an optical energy source coupled to the laser waveguide probe. The optical energy source is configured to generate a laser radiation field having energy sufficient to form a defect on a surface of the organo-mineral concretion when the laser waveguide probe is applied to the organo-mineral concretion.

The medical device also includes an electrical energy source coupled to the nanosecond electro-pulse lithotripter probe. The electrical energy source is configured to generate high-voltage nanosecond electro-pulses having energy sufficient to break the organo-mineral concretion by providing a spark electrical discharge through the organo-mineral concretion when the nanosecond electro-pulse lithotripter probe is applied to the organo-mineral concretion.

The medical device also includes a monitoring and control system configured for monitoring operation parameters and controlling operation of the device by switching operation of the device from activating of the laser waveguide probe for generating laser radiation, to activating of the nanosecond electro-pulse lithotripter probe for generating nanosecond electric pulses.

According to an embodiment, the laser waveguide probe includes one or more laser fibers for providing laser radiation to the organo-mineral concretion. Likewise, the nanosecond electro-pulse lithotripter probe includes an operating head configured to provide spark electrical discharge through the organo-mineral concretion.

According to an embodiment, a distal portion of the laser fiber of the laser waveguide probe is arranged coaxially with the operating head of the nanosecond electro-pulse lithotripter probe. For example, the laser fiber is arranged along the longitudinal axis of the combined probe. The operating head nanosecond electro-pulse lithotripter probe includes lithotripter electrodes formed as concentrically placed tubular bushings surrounding the laser fiber. Alternatively, the laser fiber has a tubular shape, and the operating head is arranged within the laser fiber.

According to an embodiment, the laser fiber of the laser waveguide probe is arranged in parallel relation to the operating head of the nanosecond electro-pulse lithotripter probe.

According to an embodiment, the combined probe includes an external sheath surrounding the laser waveguide probe and a nanosecond electro-pulse lithotripter probe.

According to an embodiment, the combined probe includes a manipulator configured to move the laser fiber and the operation head of the electro-pulse lithotripter probe independently of one another for bringing them into direct contact with the concretion.

The present invention also satisfies the aforementioned need by providing a method for destruction of the concretion utilizing the device of the present invention. The method includes generating a laser radiation field having energy sufficient to form a defect on the surface of the organo-mineral concretion; generating high-voltage nanosecond electric pulses having energy sufficient to break the organo-mineral concretion by providing the spark electrical discharge through the organo-mineral concretion; applying the laser waveguide probe to the surface of the concretion, and treating the surface with a laser radiation field to create a defect; and applying the nanosecond electro-pulse lithotripter probe to the treated surface to provide a spark electrical discharge through the concretion.

Accordingly, the method also includes manipulating the laser fiber for bringing it to a surface of the concretion for irradiating with a laser radiation field to create defects on the surface. The method also includes manipulating at least one of the electrodes of the operation head of the electro-pulse lithotripter probe for bringing the electrode into direct contact with the concretion in order to form an electrical spark with a discharge channel that is capable of generating shock waves and providing stresses that exceed the strength of the concretion material.

The use of laser radiation in the method of the present invention can form one or more defects onto the organo-mineral concretion surface that reduce the breakdown voltage provided by the nanosecond electro-pulse lithotripter (NPL) probe when the NPL probe is applied after the laser treatment, and also can increase the NPL efficacy.

Thus, the method provides destruction of the concretion by performing intra-corporeal lithotripsy through sequential treatment of a concretion with laser irradiation and then further shattering it into fragments with nanosecond electric pulses that generate substantial tensile stresses, leading to fragmentation of the concretion.

According to an embodiment of the present invention, the range of laser wavelengths lies within 0.94 μm-10.6 μm.

According to an embodiment of the present invention, the total cumulative energy during laser surface treatment lies within the range of a few Joules to several thousand Joules. Preferably, the total cumulative energy during laser surface treatment lies within the range of 15 Joules to 250 Joules.

According to an embodiment of the present invention, the surface is treated by continuous laser radiation.

According to an embodiment of the present invention, the surface is treated by pulsed laser radiation. The pulses of the pulsed laser radiation can have durations of 0.1 to 60 ms, a pulse frequency ranging from 1 to 30 Hz, and power within 0.5 to 40 W. According to an embodiment of the present invention, the energy in the laser pulse is in the range of 0.3 Joule to 5 Joules.

According to an embodiment of the present invention, the energy in the pulse of the electro-pulse lithotripter, applied to the concretion following laser treatment, is in the range of 0.1 Joule to 2 Joules.

For example, just a single electric pulse can be used. Alternatively, a train of electric pulses can be used which are applied at a frequency in the range of 1 Hz to 20 Hz.

The surface of the organo-mineral concretion can be treated with a laser and a nanosecond electro-pulse probe sequentially multiple times until total disintegration of the concretion. For example, after the initial laser treatment of the surface of the concretion to be fragmented, and its fragmentation into several parts by the nanosecond electro-pulse treatment, each fragment is treated with a laser and then fragmented into smaller fragments using the nanosecond electro-pulse lithotripter and so on, until the concretion has completely disintegrated.

There has thus been outlined, rather broadly, the more important features of the invention in order that the detailed description thereof that follows hereinafter may be better understood. Additional details and advantages of the invention will be set forth in the detailed description, and in part will be appreciated from the description, or may be learned by practice of the invention.

### BRIEF DESCRIPTION OF THE DRAWINGS

In order to understand the invention and to see how it may be carried out in practice, preferred embodiments will now be described, by way of non-limiting example only, with reference to the accompanying drawings, in which:

FIG. 1 is a schematic block diagram of the device combining electro-pulse fragmentation of organo-mineral concretions and a laser treatment of a concretion surface, according to one embodiment of the present invention;

FIG. 2 illustrates a schematic exterior view of the device for breaking a concretion in a body into smaller pieces, according to one embodiment of the present invention;

FIG. 3A illustrates a schematic side partially cross-sectional view of a distal portion of a probe of the medical device for breaking a concretion in a body into smaller pieces, according to one embodiment of the present invention;

FIG. 3B illustrates a schematic enlarged side cross-sectional view of the distal portion of the probe the medical device of FIG. 3A;

FIG. 3C illustrates a schematic transverse cross-sectional view of the distal portion of the medical device of FIG. 3A taken along the line A-A;

FIG. 3D illustrates a schematic enlarged side cross-sectional view of the distal portion of the probe the medical device according to another embodiment of the present invention;

FIG. 4A illustrates a schematic side partially cross-sectional view of a distal portion of the probe of the medical device for breaking a concretion in a body into smaller pieces, according to still another embodiment of the present invention;

FIG. 4B illustrates a schematic enlarged side cross-sectional view of the distal portion of the probe the medical device of FIG. 4A;

FIG. 4C illustrates a schematic transverse cross-sectional view of the head of the medical device of FIG. 4A taken along the line B-B;

FIG. 5A illustrates a schematic side partially cross-sectional view of a distal portion of the probe of the medical device for breaking a concretion in a body into smaller pieces, according to a further embodiment of the present invention;

FIG. 5B illustrates a schematic enlarged side cross-sectional view of the distal portion of the probe the medical device of FIG. 5A;

FIG. 5C illustrates a schematic transverse cross-sectional view of the probe head of the medical device of FIG. 5A taken along the line C-C;

FIG. 6 shows an averaged total specific volumetric energy required to break experimental samples by using various techniques;

FIG. 7 shows the dependence of specific energy required to fragment concretions for nanosecond electro-pulse treatment versus stone density and probe diameter; and

FIGS. 8A and 8B show experimental data for the cumulative energy required for fragmentation of a calculus when only a nanosecond electro-pulse treatment was applied, and when a nanosecond electro-pulse treatment was applied after the preliminary laser treatment for various types of laser and dimensions of the nanosecond electro-pulse probe.

### DETAILED DESCRIPTION OF EXEMPLARY EMBODIMENTS

The principles of the method for the medical device according to the present invention may be better understood with reference to the drawings and the accompanying description, wherein like reference numerals have been used throughout to designate identical elements. It is understood that these drawings are not necessarily to scale, are given for illustrative purposes only, and are not intended to limit the scope of the invention. Examples of constructions, materials, dimensions, and manufacturing processes are provided for selected elements. Those versed in the art should appreciate that many of the examples provided have suitable alternatives which may be utilized.

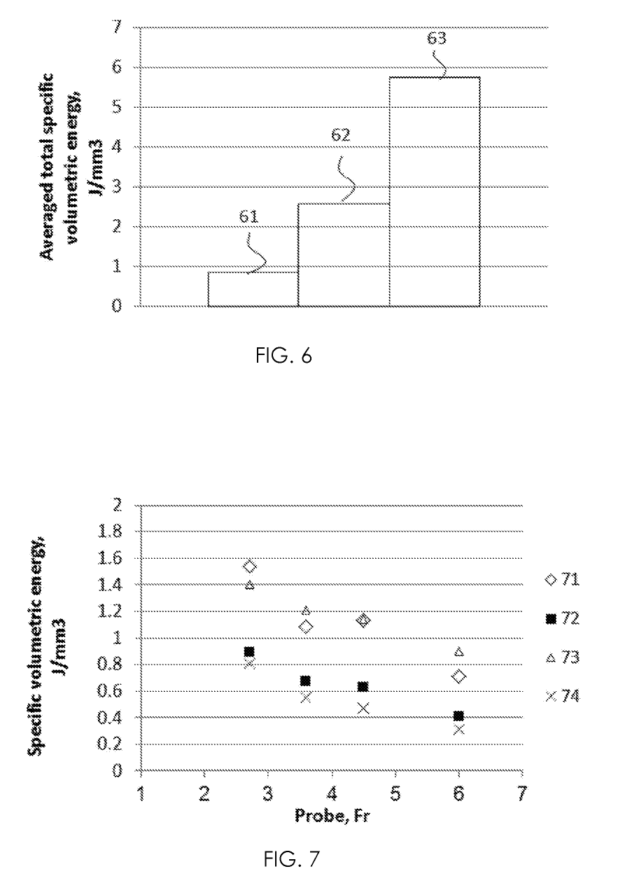

The inventors of the present application have demonstrated that NPL is the most effective method for destroying biological concretions as compared to the laser or electrohydraulic method. For example, FIG. 6 shows an averaged total specific volumetric energy required to break experimental samples of organo-mineral concretions for the following techniques: NPL—nanosecond electro-pulse lithotripter technique (indicated by a reference numeral 61), EHL—electrohydraulic lithotripter technique (indicated by a reference numeral 62); and LL—Ho:YAG laser lithotripter technique (indicated by a reference numeral 63). As can be seen, the specific volumetric energy required for destroying concretions is less than 1 J/mm³ for the NPL technique. This energy is greater than 2 J/mm³ for the EHL technique and greater than 5 J/mm³ for the laser lithotripter technique.

Moreover, it was also found that the effectiveness of the NPL method depends not only on the density and structure of the concretion, but also on the dimension of the NPL probe. FIG. 7 shows the dependence of specific energy required to fragment concretions for the NPL technique versus stone density and probe diameter. Electrical pulses of energy of 1 Joule and 0.8 Joule were used when NPL was used for destruction of soft and hard organo-mineral concretions. Symbols indicated by a reference numeral 71 correspond to the experiments for destruction of hard concretions using electrical pulses of energy of 1 Joule. Symbols indicated by a reference numeral 72 correspond to the experiments for destruction of soft concretions using electrical pulses of energy of 1 Joule. Symbols indicated by a reference numeral 73 correspond to the experiments for destruction of hard concretions using electrical pulses of energy of 0.8 Joule. Symbols indicated by a reference numeral 74 correspond to the experiments for destruction of soft concretions using electrical pulses of energy of 0.8 Joule.

Probes having diameters of 4.5 Fr (French) and 6 Fr were used in the experiments.

As can be seen in FIG. 7, while working with probes having (i.e. 3.6 Fr), which are typically used for managing blood vessels, kidney stones, etc., as well as when stone density is increased, fragmentation energy and time are relatively greater than when working with probes having large diameter (i.e. 6 Fr). This is an undesirable effect, which complicates the procedure and may lead to tissue damage due to the increase in fragmentation energy required to achieve a greater effect for probes with small diameters.

In this connection, the inventors of the present application contemplated a method that allows increasing efficacy of the NPL technique. Thus, according to an embodiment of the present invention, at the first stage, a surface of the concretions is irradiated by a laser beam, in order to create defects on the surface. After treatment with laser radiation, fragmentation of the concretion can be culminated by applying the nanosecond electro-pulse lithotripsy technique, that provides a spark discharge through the concretion, resulting in destruction of the concretion into small pieces. Such a combined treatment can provide the possibility to fragment even large and dense concretions by applying insignificant cumulative energy for a short time period.

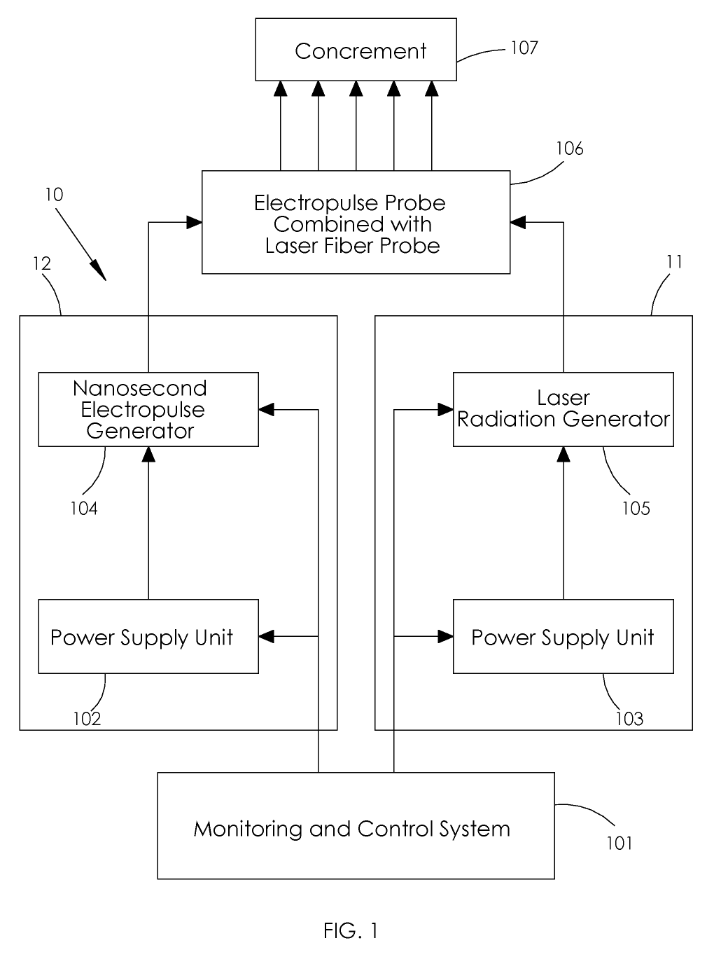

FIG. 1 illustrates a schematic block diagram of a device 10 for breaking an organo-mineral concretion 107 into smaller pieces by combining a laser treatment and electro-pulse breakdown techniques, according to one embodiment of the present invention. The device for breaking a concretion into smaller pieces includes a probe 106 which has two parts, such as a laser waveguide probe (not shown in FIG. 1), which has one or more laser fibers, and a nanosecond electro-pulse lithotripter probe (not shown in FIG. 1) which has electrodes.

Since the probe 106 includes both a laser waveguide probe and a nanosecond electro-pulse lithotripter probe, it is also referred to as a “combined probe”. The combined probe 106 is coupled to an optical energy source 11 including a laser radiation generator 105 optically coupled to the laser waveguide probe and configured for generating laser radiation. The combined probe 106 is also coupled to an electrical energy source 12 including a nanosecond electro-pulse generator 104 electrically coupled to the nanosecond electro-pulse lithotripter probe and configured for generating high-voltage pulses. According to the present invention, laser radiation is required to form defects on the surface of the concretion 107. In turn, high-voltage pulses are required to provide a spark discharge through the concretion 107. Therefore, such high-voltage pulses should have energy sufficient for fragmentation of the concretion into smaller pieces. The nanosecond electro-pulse generator 104 and the laser radiation generator 105 are powered by corresponding power supply units 102 and 103, correspondingly.

The device 10 also includes a monitoring and control system 101 configured for controlling, monitoring and selecting operating parameters of the device 10. The monitoring and control system 101 is configured also for switching operation of the device 10 from providing laser radiation to providing nanosecond electric pulses for transferring the energy of laser radiation and nanosecond electric pulses to the concretion 107, correspondingly. In operation, the monitoring and control system 101 provides monitoring and controlling of the device 10 that allows setting the required parameters required for the operation of both the laser radiation generator 105 of the optical energy source 11 and the nanosecond high-voltage generator 104 of the electrical energy source 12.

The monitoring and control system 101 performs supervision of operation of the laser radiation generator 105 and the nanosecond high-voltage generator 104, monitoring the values of the laser radiation pulse parameters and the nanosecond electric pulses along with other operating parameters for optimum implementation of the method for breaking organo-mineral concretions into smaller pieces. Optimum implementation is achieved through combining the operations of the laser radiation generator 105 and the nanosecond electro-pulse generator 104 to transfer energy to the concretion 107 for its fragmentation upon application of optic and electric energy pulses.

According to an embodiment, the device 10 can include a manipulation system (not shown in FIG. 1) configured for manipulating the combined probe 106.

According to an embodiment, treatment of an organo-mineral concretion with the combined probe 106 includes pre-treatment of a surface of the concretion with a laser radiation field that is followed by fragmentation of the concretion by applying the nanosecond electro-pulse lithotripsy technique. Thus, the device 10 combines the generation of a laser radiation field and nanosecond electric pulses in order to implement fragmentation of organo-mineral concretions.

According to another embodiment, fragmentation of a concretion begins with treatment by the nanosecond electro-pulse lithotripsy technique so as to apply electric spark discharge through the concretion to form preliminary defects in the calculus. The electric spark discharge treatment is then followed by formation of further external damage by using the laser radiation field. Finally, fragmentation culminates in secondary application of the nanosecond electro-pulse lithotripsy technique.

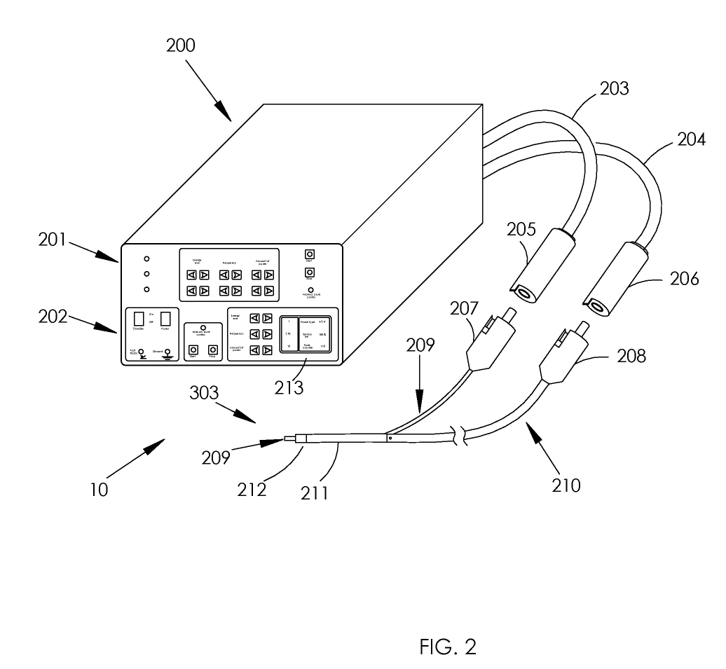

FIG. 2 illustrates a schematic view of the device 10 for breaking a concretion in a body into smaller pieces, according to one embodiment of the present invention.

Referring to FIG. 1 and FIG. 2 together, the device 10 includes a housing module 200 for housing the monitoring and control system 101, optical energy source 11 and an electrical energy source 12. The device 10 also includes a nanosecond electro-pulse generator cable 204 electrically coupled to a nanosecond electro-pulse lithotripter probe 210 and a laser waveguide 203 optically coupled to a laser waveguide probe 209. The lithotripter probe cable 204 and the laser waveguide 203 are configured for transferring energy from the generators of nanosecond pulses and the laser radiation field to the concretion (not shown) to be fragmented. The housing module 200 also includes monitoring screens 213, and optical and electrical pulse parameter selectors 201 and 202, which allow the operator to supervise the lithotripsy process and monitor the operating parameters for both laser treatment and electro-impulse lithotripsy treatment.

During operation of the device 10, the level of the optical pulse energy and electrical pulse energy can be monitored and controlled; the number of pulses delivered, the frequency of the pulses, the number of pulses in each series of energy application, and the cumulative energy delivered to the calculus by each part of the device, and the power of the delivered pulses, can also be monitored. It should be understood that there can also be other parameters of the treatment process that can be controlled by the monitoring and control system 101 and displayed on the screens 213.

Control signals from the monitoring and control system 101 are transferred to the power supply units 102, 103 to activate operation of the laser radiation generator 105 and the nanosecond electric pulse generator 104. Nanosecond high-voltage pulses and laser radiation pulses are relayed to the combined probe 106, which includes both a nanosecond electro-pulse lithotripter probe 210 and a laser waveguide probe 209. The combined probe (106 in FIG. 1) is indicated by a reference numeral 211 in FIG. 2.

A head 212 of the combined probe 211 at a distal end 303 includes electrodes (not shown) of the nanosecond electro-pulse lithotripter probe 210 and one or more laser fibers of the laser waveguide probe 209.

The lithotripter electrodes and the laser fiber(s) can be moved independently relative to one another, thus enabling two types of treatments, for example, a preliminary treatment of a concretion with laser radiation field and a further fragmentation of the concretion by using the lithotripter probe sequentially and independently of one another.

According to an embodiment, the monitoring and control system 101 of the device 10 includes a laser radiation parameter selector (indicated by a reference numeral 201) and a nanosecond electro-pulse lithotripter parameter selector (indicated by a reference numeral 202). In operation, the pulse parameter selectors 201 and 202 coordinate operation of the monitoring and control unit, that in turn coordinates operation of the optical energy source 11 and the electrical energy source 12. The nanosecond electro-pulse generator 104 and the laser radiation generator 105 receive corresponding control signals from the monitoring and control system 101. In turn, the monitoring and control system 101 sets the values of the required parameters of laser radiation energy and nanosecond high-voltage pulses using the pulse parameter selectors 201 and 202. The electro-pulse energy and the laser light energy from the generators 104 and 105, correspondingly, is transferred through the power transmission lines to the combined probe 106.

According to an embodiment, a laser waveguide 203 is used in order to transmit laser radiation, whereas an electro-pulse generator cable 204 is used in order to transmit nanosecond high-voltage electric pulses. The cable 204 can, for example, include a coaxial cable and/or a twisted pair two-wire. The laser waveguide 203 and the electro-pulse generator cable 204 are flexible, elastic elements that can be moved independently, relative to one another.

The laser waveguide 203 and the electro-pulse generator cable 204 have corresponding connectors, such as a laser waveguide connector 205, and a nanosecond electro-pulse cable connector 206. The connectors 205 and 206, in turn, are linked, correspondingly, to a waveguide connector 207 associated with the laser waveguide probe 209 and to a cable connector 208 associated with the nanosecond electro-pulse lithotripter probe 210 at their proximal ends. It should be noted that in the description and claims that follow, the terms “proximal” and “distal” are used with reference to the operator of the medical device.

The connectors 205-208 are matched correspondingly to one another to avoid losses during signal transmission. Thus, for transferring nanosecond electric pulses, the connectors 206 and 208 should have at least the same wave impedance, also matched to the cable 203 and the cable (not shown) of the nanosecond electro-pulse lithotripter probe 210. By the same token, the connectors 205 and 207, the waveguide 203 and the laser fiber(s) 213 of the laser waveguide probe 209 should have the same transferring properties for the selected wavelength.

As will be described hereinbelow in detail, the laser fiber(s) of the laser waveguide probe 209 and the cable of the nanosecond electro-pulse probe 210 are combined together at their distal ends under a common outer sheath (not shown) to form the combined probe 211. The nanosecond electro-pulse probe 210 is equipped with an operating head 212 including potential and ground electrodes (not shown) coupled to the cable of the nanosecond electro-pulse probe 210.

For example, in operation, during urological procedures, the combined probe 211 of the device 10 can be placed into a urological endoscope (not shown). The distal end 213 of the laser fiber(s) of the laser waveguide probe 209 or the electrodes of the operating head 212 nanosecond electro-pulse probe nanosecond arranged at the distal end of the nanosecond electro-pulse probe 210 can be brought to the concretion in order to apply fragmentation energy thereto.

The total length of the combined probe 211 together with the laser waveguide probe 209 and the electro-pulse lithotripter probe 210 can, for example, be within 400 mm to 2500 mm, however, depending on the clinical requirements, these values can be varied upward or downward. A length of the operating head 212 at the distal end of the nanosecond electro-pulse lithotripter probe 210 can, for example, be in the range of 5 mm to 20 mm, and the outer diameter of the distal end of the combined probe can, for example, be from 0.6 mm to 5 mm, but these values can be also changed depending on the specific clinical application.

According to one embodiment, the combined probe 211 is made as a fixed unit including laser waveguide probe 209 and the electro-pulse lithotripter probe 210, which are immovable relative to one another.

According to another embodiment, the laser waveguide probe 209 can be movable relative to the electro-pulse lithotripter probe 210.

According to one embodiment, in operation, the start of fragmentation of the calculus or other concretion is carried out with application of laser energy emitted from the laser fiber of the laser waveguide probe 209 to the concretion surface in order to generate initial surface defects thereon. After application of laser energy, the operating head 212 of the nanosecond electro-pulse lithotripter probe 210 can be brought to the surface of the concretion damaged by the laser radiation to apply electric spark discharge through the concretion, that completes fragmentation of the concretion.

According to another embodiment, the fragmentation of a calculus starts from the treatment by the nanosecond electro-pulse lithotripter probe 210 so as to apply electric spark discharge through the concretion to form preliminary defects in the calculus. The electric spark discharge treatment is then followed by formation of further external damage by using the laser radiation emitted from the laser fiber(s) of the laser waveguide probe 209, and then the fragmentation culminates in secondary application of the nanosecond electro-pulse lithotripter probe 210.

According to an embodiment, the monitoring and control system 101 can also be programmed for estimation of the operational life of the device 10 to provide information on the remaining operational life of the lithotripter probe and the laser waveguide probe. When desired, information about the amount of energy that has passed through the lithotripter probe and through the laser waveguide probe can also be provided.

According to an embodiment, the monitoring and control system 101 is also configured for monitoring the operational life of the electro-pulse lithotripter probe and the laser waveguide, and notifies the operator in advance of expiration of the operating life.

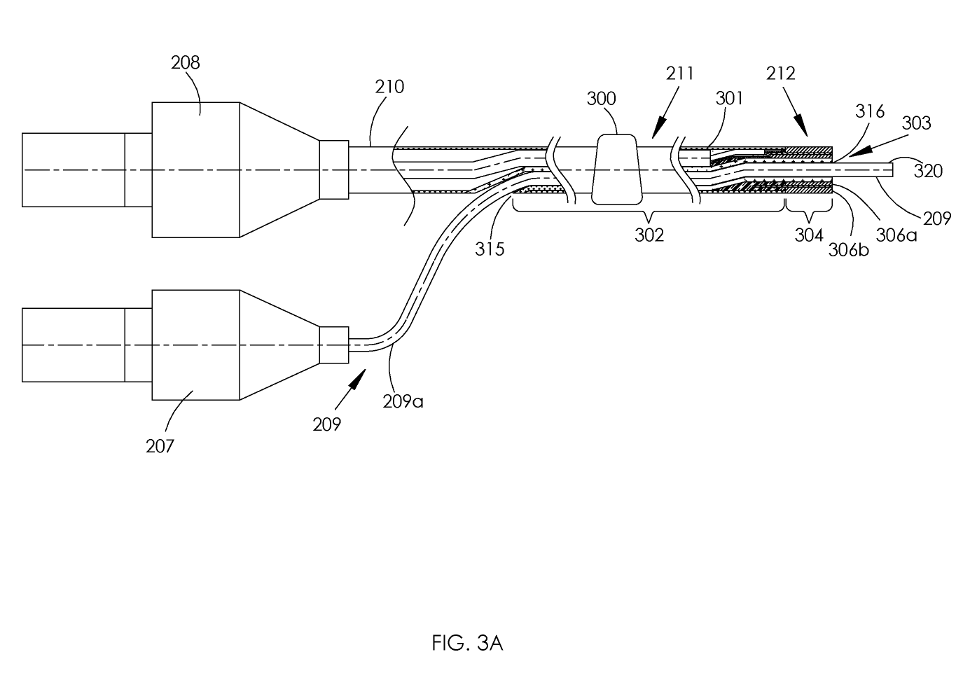

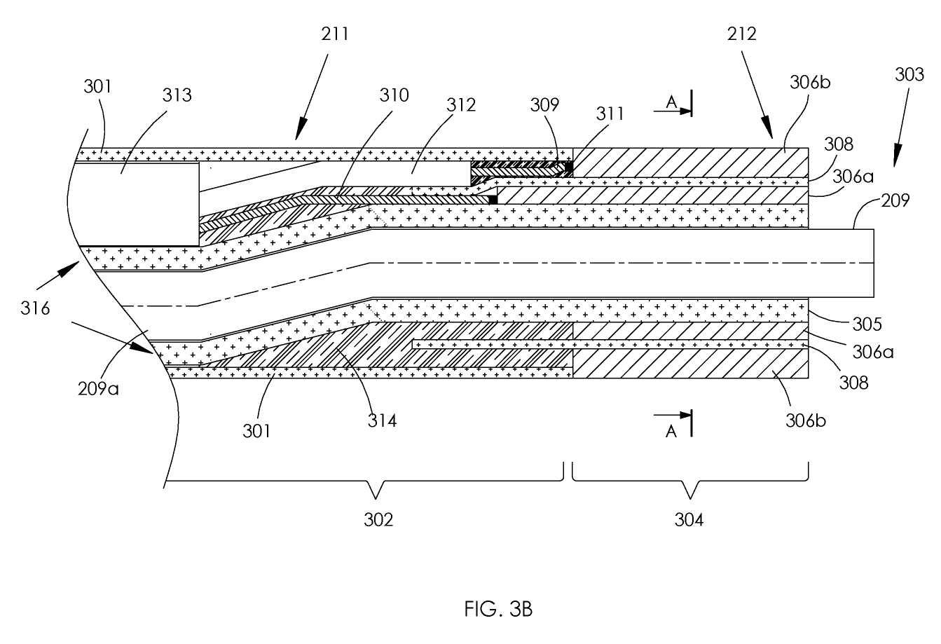

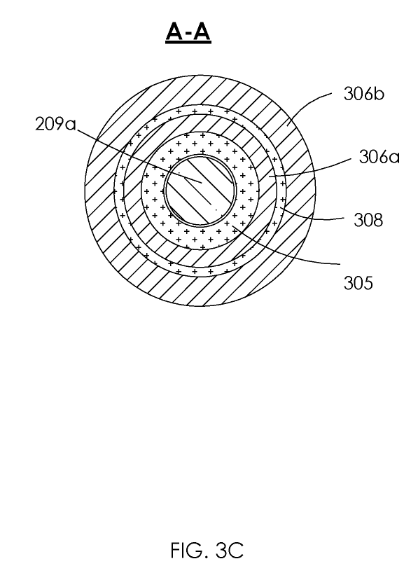

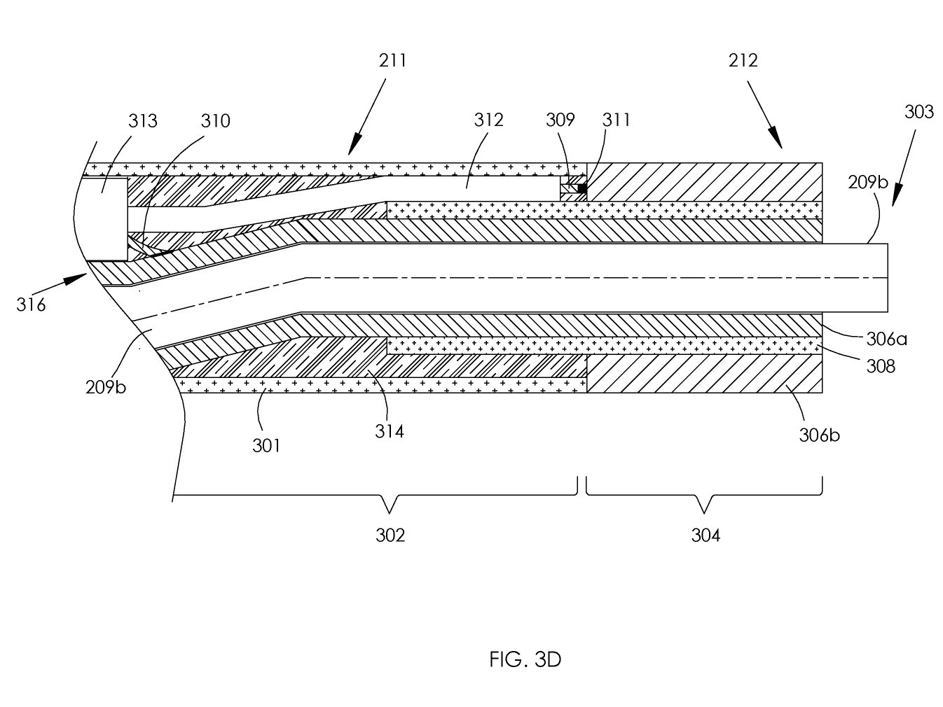

Referring to FIG. 3A, a schematic side partially cross-sectional view of the combined probe 211 of the medical device 10 for breaking a concretion into smaller pieces is illustrated, according to one embodiment of the present invention.

The combined probe 211 includes the laser waveguide probe 209 and the nanosecond electro-pulse lithotripter probe 210 connected to the generators (11 and 12 in FIG. 1) of laser radiation and high-voltage nanosecond pulses, correspondingly via connectors 207 and 208. The combined probe 211 also includes an external sheath 301 at a distal portion of the combined probe 211. The distal portion of combined probe 211 has zones 302 and 304, and a distal end 303. The external sheath 301 of the combined probe 211 can be made of various materials, such as polyimide, braided reinforced polyimide, polyvinyl chloride, silicone rubber, nitinol, nylon, polyurethane, polyethylene terephthalate (PETE) latex, and thermoplastic elastomers, etc.

According to the embodiment shown in FIG. 3A, the laser waveguide probe 209 includes a laser fiber 209a arranged along the longitudinal axis of the combined probe 211. A distal portion 320 of the laser fiber 209 is located coaxially with the operating head 212 of the electro-pulse lithotripter probe 210 within the external sheath 301 of the combined probe 211. The laser fiber 209a is introduced in the external sheath 301 into a lumen 316 of the combined probe 211 through an enter opening 315, and is then located in zones 302, 304 up to the distal end 303 of the combined probe 211. In operation, the laser fiber 209 can exit out of the external sheath 301 from the distal end 303 towards the concretion.

The enter opening 315 can, for example, be located 15 cm to 120 cm away from the distal end 303 of the combined probe 211. However, it should be understood that this distance can be varied in any direction, depending on the clinical application of this device.

The operating head 212 of the electro-pulse lithotripter probe 210 is located in the zone 304 and includes electrodes (306a and 306b). Thus, the laser fiber and the electrodes of the nanosecond are arranged in the combined probe 211 under the common external sheath 301.

According to the embodiment shown in FIG. 3A, the combined probe 211 also includes a manipulator 300 located in the zone 302. The manipulator 300 is configured to permit movement of the laser fiber 209a and the operation head 212 of the electro-pulse lithotripter probe 210 independently of one another. It should be understood that the manipulator 300 is designed to simplify the procedure of movement of the fiber 209a and the operation head 212 relative to one another, but such movement can also be carried out without such a special manipulator. The operation can be carried out by moving the laser fiber 209a inside of the lumen 316 which is located coaxially in the probe 210 or through the movement of the head 212 of the probe 210 itself towards the concretion.

FIG. 3B shows a detailed enlarged view of the combined probe 211 of FIG. 3A. As described above, the laser fiber 209a enters into the electro-pulse lithotripter probe 210 in the enter opening (315 in FIG. 3A) and is located within the operating head 212 of the electro-pulse lithotripter probe along the longitudinal axis of the combined probe 211. Thus, the electrodes 306a and 306b surround the laser fiber 209a.

While moving along the lumen 316, the laser fiber 209a can leave the distal end 303 of the combined probe 211 and can be brought into contact with the concretion (not shown) to irradiate its surface and create preliminary cracks. Then, the laser fiber 209a can be retracted into the probe 211. After treatment of the concretion with laser radiation, the electrodes 306a and 306b of the nanosecond electro-pulse lithotripter probe head 212 can be brought into contact with the concretion for its fragmentation.

According to one embodiment, the laser fiber 209a is separated from the operating elements of the nanosecond electro-pulse lithotripter probe head 212 by the lumen 316 which forms a dielectric insulator layer 305 between the electrodes 306a and 306b and the fiber 209a , thus preventing creation of potentials on the laser fiber. When desired, the lumen 316 can be filled with a dedicated insulation material to form the insulation layer 305. The insulation layer 305 can be made of various dielectric elastic materials, such as polyvinyl chloride, rubber, polyimide, braided reinforced polyimide, silicone rubber, nitinol, nylon, polyurethane, polyethylene terephthalate (PETE) latex, and thermoplastic elastomers, etc.

Referring to FIG. 3C, the described embodiment of the combined probe 211 is also represented on the A-A cross-section. The electrodes 306a and 306b of the operating head 212 of the nanosecond electro-pulse lithotripter probe are located around the insulation layer 305. The electrode 306a (internal electrode) and the electrode 306b (external electrode) can, for example, be formed as concentrically placed tubular bushings, separated by a tubular insulator layer 308 placed between them. The electrodes 306a and 306b can, for example, be made from an electrically conductive material with a relatively high conductivity and can be manufactured from metals of various groups such as, for example, steel or multicomponent alloys, preferably from stainless steel or cobalt-nickel alloys. The insulator layer 308 has a high dielectric strength and is made, for example, from polyimide, ceramics, nanoceramics, etc. When desired, the insulator layer 308 can be made from several insulating bushings made from dielectric material that can be connected together with glue to increase dielectric breakdown resistance and increase erosion resistance.

During fragmentation of a concretion, the operating action includes application of laser energy to the concretion by using the laser fiber 209a that emits laser radiation, as well as application of the electric discharge occurring between the electrodes 306a and 306b through the concretion.

Turning back to FIGS. 3A and 3B together, electric pulses are transferred to the electrodes 306a and 306b from the nanosecond electro-pulse generator (104 in FIG. 1) through the cable 312. As shown in FIGS. 3A and 3B, the coaxial cable 312 can be connected to one of the cylindrical electrodes (e.g., to the electrode 306b that is external electrode) via its central (potential) core wire 309 and to the other cylindrical electrode 306a via the cable braid shield 310. It should be understood that, when desired, the connection of the cable 312 to the electrodes 306a and 306b can also be reversed, that is, the cable shield can be connected to the external electrode 306b, whereas the central (potential) cable core can be connected to the electrode 306a. Furthermore, the cable shield can be grounded or zeroed out. The electrical cable 312 has an insulation layer between the core 309 and the cable shield 310 that is made of a dielectric material having high dielectric strength, for example, a type of Teflon (polytetrafluoroethylene), etc. In making the above-mentioned connection, the core 309 of the cable 312 with its insulation and the cable braid shield 310 are pre-stripped from an external jacket 313 of the coaxial cable 312.

Connection of the core 309 and the cable shield 310 to the electrodes 306a and 306b of the nanosecond electro-pulse lithotripter head 212 can be made by several methods used in bounding of electrical wires, but it is preferable that they are linked with soldering 311. In so doing, the connection points must be spatially separated, at least at a distance no less than 2 mm to reduce the probability of breakdown at the point of the cable connection to the electrodes when high-voltage pulses are transmitted.

According to an embodiment, a twisted pair cable can also be used instead of a coaxial cable.

The voids formed during assemblage of the combined probe 211 can be filled with glue 314. It is preferable to use high-strength glues with good dielectric properties, for example, epoxy adhesive.

The combined probe 211 is arranged such that, during the lithotripsy procedure, the laser waveguide probe 209 can be placed in various positions relative to the operating head 212 of the nanosecond electro-pulse lithotripter 210 while being moved either with the manipulator 300, or manually. For example, the laser fiber 209a can project over the head 212 of the nanosecond electro-pulse lithotripter, such that that its position is specific to the start of the operation when energy from the laser generator is applied through the fiber to the calculus to form surface defects. Alternatively, the laser fiber 209a can also be located inside the nanosecond electro-pulse lithotripter head, and this position is also acceptable when the laser waveguide is not used, while the nanosecond electro-pulse lithotripter probe is only operating in order to fragment the concretion after its laser treatment.

One more alternative embodiment of the combined probe 211a is illustrated in FIG. 3D. The combined probe 211a shown in FIG. 3D differs from the combined probe 211 shown in FIGS. 3A-3C by the fact that the laser fiber 209b initially has a dielectric outer sheath (not shown) that is tightly fitted on the outer surface of the laser fiber, thus forming an integral unit with the fiber. According to this embodiment, the lumen 316 surrounding the laser fiber 209a is filled with a conductive material. In this case, it is connected directly to one of the conductors of the coaxial cable (e.g., to the cable shield 310, as shown in FIG. 3D), and thus serves as one of the electrodes (i.e. electrode 306a in FIG. 3D) of the nanosecond electro-pulse lithotripter.

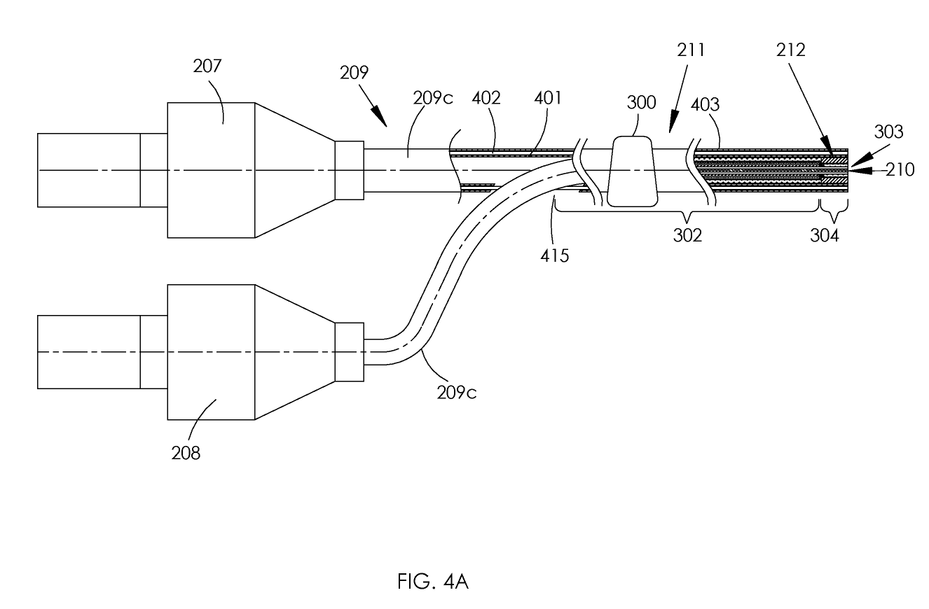

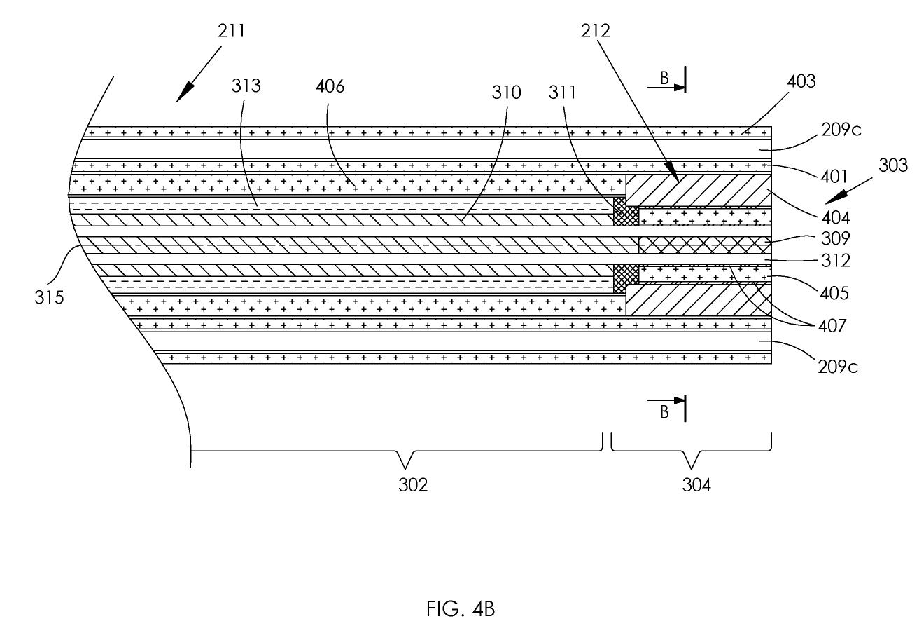

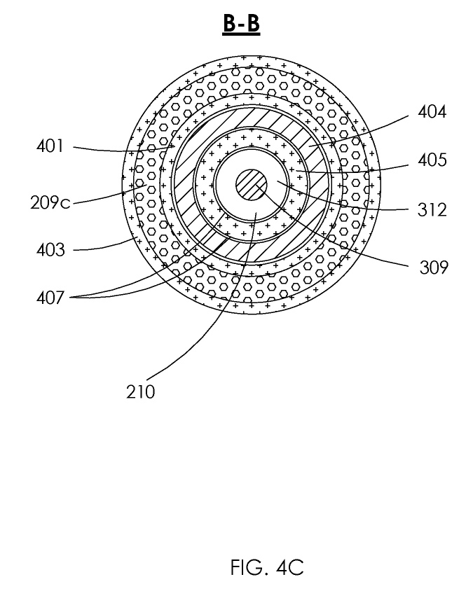

FIGS. 4A-4C show a combined probe 211c of the nanosecond electro-pulse lithotripter probe 210, according to a further embodiment of the present invention. In this embodiment, the laser fiber 209a of the laser waveguide probe 209 and the operating head 212 of the nanosecond electro-pulse lithotripter probe 210 are also located in axial alignment. However, the combined probe 211c shown in FIGS. 4A-4C differs from the combined probe 211 shown in FIGS. 3A-3C by the fact that the operating head 212 of the nanosecond electro-pulse lithotripter probe is located within a tubular laser fiber 209c.

According to this embodiment, the laser fiber 209c is shaped as a hollow tube, and the nanosecond electro-pulse lithotripter probe 210 the nanosecond electro-pulse lithotripter probe 210 can be introduced inside of a hollow lumen 401 of the laser fiber 209c.

In this case, the internal wall of the tubular laser fiber 209c serves as a wall of the lumen 401. An external wall of the laser fiber 209c can be surrounded by an external sheath 403 which can be an external jacket of the laser fiber 209c.

In this embodiment, the operating head 212 of the nanosecond electro-pulse lithotripter probe 210 is introduced in the laser waveguide 209c in an enter area 415 and is then located in zones 302, 304, near the distal end 303 of the combined probe 211.

Thus, the laser waveguide probe 209 and the electro-pulse lithotripter probe 210 are aggregated in a combined probe 211c under the common external sheath 403.

The external sheath 403 can be made from various elastic dielectric materials. Examples of suitable materials include, but are not limited to, polyimide, polyvinyl chloride, rubber, silicone rubber, nitinol, nylon, polyurethane, polyethylene terephthalate (PETE) latex, and thermoplastic elastomers.

As shown in detail in FIG. 4B, the nanosecond electro-pulse lithotripter probe 210 includes a coaxial cable connected to a central core electrode 309 and a tubular electrode 404 of the operating head 212 which are located in the zone 304 of the combined probe 211c. The tubular electrode 404 of the operating head 212 is in the form of a cylindrical bushing concentrically located within the lumen 401, whereas the central core 315 of the coaxial cable of the electro-pulse lithotripter probe 210 can be connected to the electrode 309. In this case, the electrode 404 can be connected to the cable shield 310. Connection of the electrodes to the coaxial cable can be made by various methods used in the bounding of electric wires, such as soldering, welding etc. In particular, connection of the electrode 404 to the cable shield 310 can be made by soldering via layer 311.

The central core electrode 309 is insulated from the tubular electrode 404 by an insulator layer 312 that can, for example, be the insulator layer of the coaxial cable. An additional insulator layer 405 having high dielectric strength (e.g., made from polyimide) can also be arranged in the zone 304 between the central core electrode 309 and a tubular electrode 404 of the operating head 212. The insulator layer 405 can be glued to the tubular electrode 404 by a glue layer 407. Alternatively, the insulator 405 can be manufactured from a set of tubes affixed to each other with glue in order to increase dielectric strength. The glue layer 407 must have suitable dielectric properties and mechanical strength. For example, an epoxy-based glue can be used for the glue layer 407.

In order to impart greater rigidity to the structure during assemblage of the combined probe, a sealing layer 406 made from elastic dielectric material, e.g., from polyimide, Teflon or other polymeric material, can be arranged in the cavity 401 between an external jacket 313 of surrounding of the cable shield 310 and the internal wall the tubular fiber 209c. In operation, using the manipulator 300 or manually, either the fiber 209c or the lithotripter electrodes 404 and 309 can be brought into contact with the concretion independently of one another.

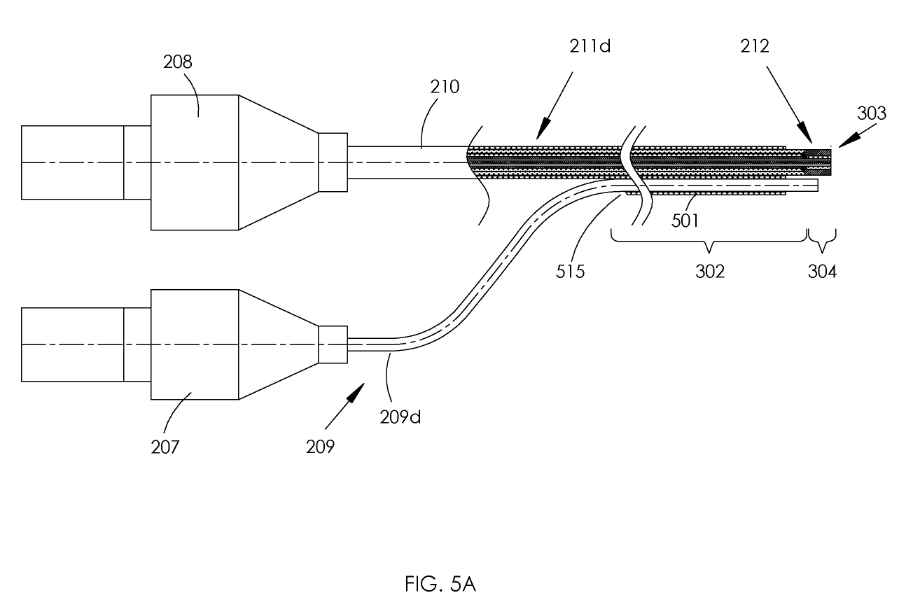

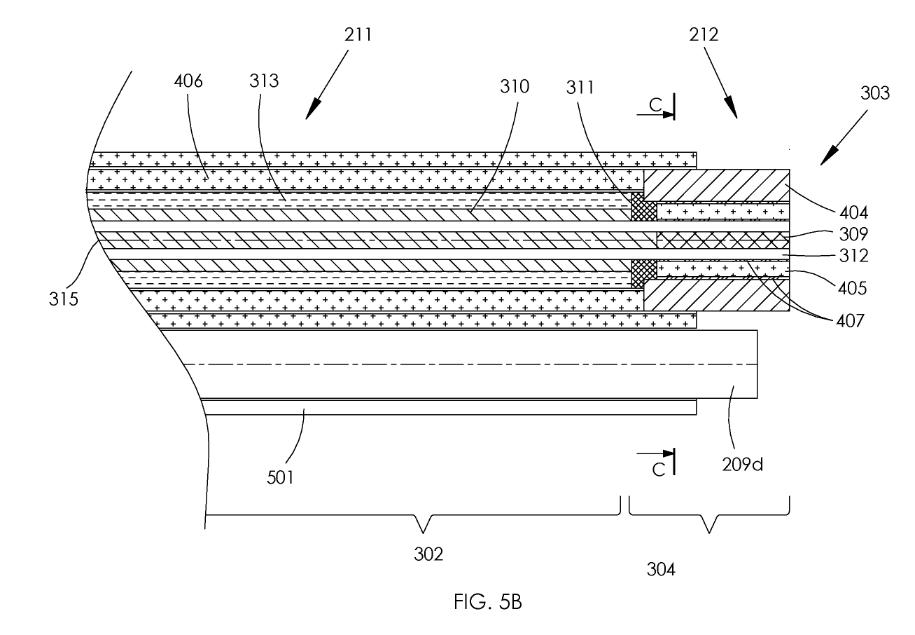

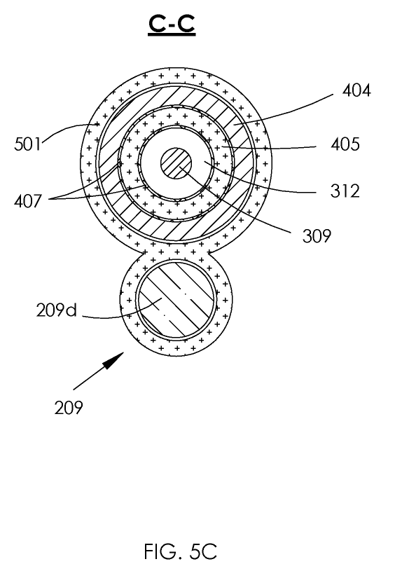

Referring to FIGS. 5A-5C, a combined probe 211d is shown, according to a further embodiment of the present invention. According to this embodiment of the combined probe 211d differs from the combined probe shown in FIGS. 3A-3C and 4A-4C by the fact that a laser fiber 209d of the laser waveguide probe 209 is arranged in parallel relation to the operating head 212 of the nanosecond electro-pulse lithotripter probe 210 under a common external sheath 501.

FIG. 5B represents a detailed enlarged view of the combined probe 211d in zones 302 and 304. The laser fiber 209d is introduced in the external sheath of the combined probe 211d in an opening 515 of the common external sheath 501. The common external sheath 501 of the combined probe can be made of various materials including, but are not limited to polyimide, braided reinforced polyimide, polyvinyl chloride, silicone rubber, nitinol, nylon, polyurethane, polyethylene terephthalate (PETE) latex, and thermoplastic elastomers.

According to this embodiment, the nanosecond electro-pulse lithotripter probe 210 includes a coaxial cable connected to a central core electrode 309 and a tubular electrode 404 of the operating head 212 which are located in the zone 304 of the combined probe 211d. The tubular electrode 404 of the operating head 212 is in the form of a cylindrical bushing connected to the cable shield 310. The central core 315 of the coaxial cable of the electro-pulse lithotripter probe 210 is connected to the electrode 309. The connection of the electrodes 309 and 404 to the coaxial cable can be made by various methods used in the bounding of electric wires, such as soldering, welding etc. In particular, the connection of the electrode 404 to the cable shield 310 can be made by soldering via the layer 311.

The central core electrode 309 is insulated from the tubular electrode 404 by an insulator layer 312 that can, for example, be an insulator layer of the coaxial cable. An additional insulator layer 405 having high dielectric strength (e.g., made from polyimide) can also be arranged in the zone 304 between the central core electrode 309 and a tubular electrode 404 of the operating head 212. The insulator layer 405 can be glued to the tubular electrode 404 by a glue layer 407. Alternatively, the insulator 405 can be manufactured from a set of tubes affixed to each other with glue in order to increase dielectric strength. The glue layer 407 must offer good dielectric properties and mechanical strength. An epoxy-based glue can be used for the glue layer 407.

The laser fiber 209d of the laser waveguide probe 209 is arranged in a parallel relation to the lithotripter electrodes 404 and 309 of the operating head 212 under the common external sheath 501. In operation, either the fiber 209d or the lithotripter electrodes 404 and 309 can be brought into contact with the concretion independently of one another.

A cross-section of the combined probe 211d along the C-C line is presented in FIG. 5C.

According to another aspect of the present invention, there is provided a novel method for breaking a concretion into smaller pieces by using the combined probe of the present invention. The method includes generating laser radiation field having energy sufficient to form defects on a surface of the concretion, and applying the laser radiation field to the surface of the concretion for treating the surface with a laser radiation field to create a defect. The method also includes generating nanosecond high-voltage pulses having energy sufficient to create a spark discharge through the concretion and applying the spark discharge to the concretion in the area of the defect created by the laser radiation field for fragmenting the concretion into smaller pieces.

According to an embodiment, treatment of an organo-mineral concretion with the combined probe includes pre-treatment of a surface of the concretion with a laser radiation field in order to generate initial surface defects thereon. The laser radiation treatment is followed by fragmentation of the concretion by applying the nanosecond electro-pulse lithotripsy technique.

According to another embodiment, fragmentation of a concretion begins with treatment by the nanosecond electro-pulse lithotripsy technique so as to apply electric spark discharge through the concretion to form preliminary defects in the concretion. The electric spark discharge treatment is then followed by formation of further external damage by using a laser radiation field. Finally, fragmentation culminates in secondary application of the nanosecond electro-pulse lithotripsy technique.

It was found that wavelength of the laser radiation may be in the ultraviolet, visible, or infrared range of the spectrum. However, in terms of safety of laser radiation, wavelengths in the range of 0.94 μm to 10.6 μm are preferable.

A total cumulative energy during laser surface treatment can, for example, be in the range of a few Joules to several thousand Joules, and preferably in the range of 15 Joules to 250 Joules.

According to one embodiment, the laser radiation field is a continuous laser radiation field.

According to another embodiment, the laser radiation field is a pulsed laser radiation field. In this case, a duration of the pulses (pulse width) of the pulsed laser radiation can be in the range of 0.1 ms to 60 ms, a pulse frequency can be in the range of 1 Hz to 30 Hz and a pulse energy in the is in the range of 0.3 Joule to 5 Joules. A power of the laser radiation field can be in the range of 0.5 W to 40 W.

According to one embodiment, a duration of the high-voltage nanosecond pulses generated by the electro-pulse lithotripter probe and applied to the concretion following laser treatment can be in the range of 100 nanoseconds to 1000 nanoseconds with a pulse rise time (pulse front) in the range of 1 ns to 50 nanoseconds. A magnitude of the pulses can be in the range of 5kV to 20 kV. An energy of the high-voltage nanosecond pulses can be in the range of 0.05 Joule to 10 Joules, preferably in the range of 0.1 Joule to 2 Joules. A single high-voltage nanosecond pulse or a train of high-voltage nanosecond pulses can be applied to the concretion. When a train of high-voltage nanosecond pulses is used, frequency of the pulses (pulse rate) can, for example, be in the range of 1 Hz to 30 Hz, and preferably in the range of 3 Hz to 20 Hz.

As described above, preliminary laser treatment of a concretion surface provides defects on the concretion surface. A further treatment of the concretion by application of the nanosecond electro-pulse lithotripter (NPL) technique to the locations with the laser-generated defects provides electric discharge and breakdown through the bulk of the concretion, resulting in fragmentation of the calculus. If the concretion is fragmented into several relatively large pieces, then these large concretion pieces may again be treated with a laser radiation field and spark discharge through the concretions, if required. Thus, the treatment may run sequentially, as long as required, until the concretion has completely disintegrated.

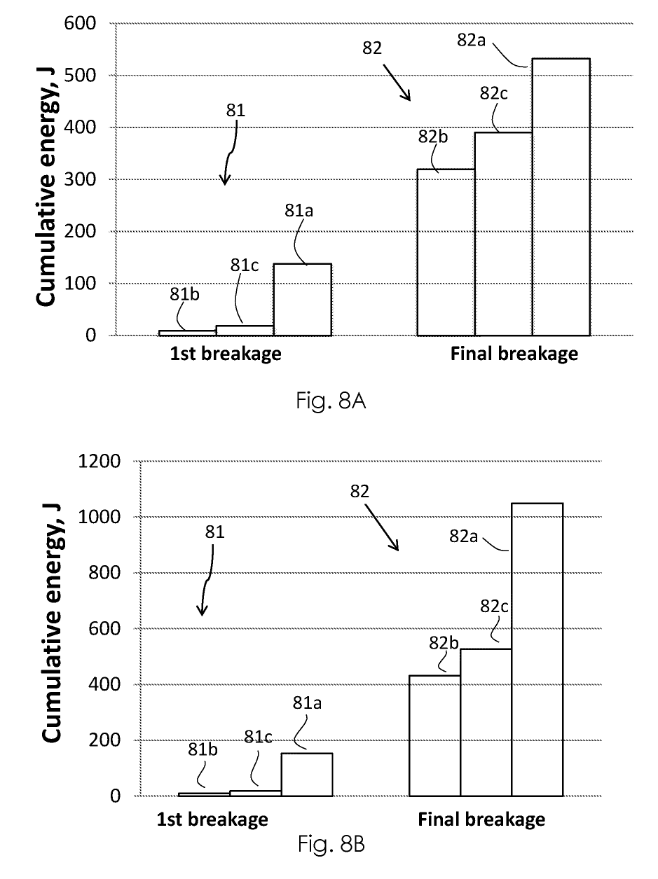

FIGS. 8A and 8B show examples of experimental data for the cumulative energy required for a first breakage of a concretion (the experimental data are indicated by a reference numeral 81), and the experimental data for the cumulative energy required for total disintegration of the stone pieces which remain after the first breakage (the experimental data are indicated by a reference numeral 82). The experimental data are presented for the cases when only a NPL treatment was applied (the experimental data are indicated by reference numerals 81a and 82a), and when a NPL treatment was applied after the preliminary laser treatment with a laser radiation field (81b, 81c and 82b, 82c). The results are presented for various types of lasers.

Specifically, FIG. 8A and 8B corresponds to the treatment with diode laser and NPL probe (the experimental data are indicated by reference numerals 81b and 82b); and to the treatment with Ho:YAG laser and NPL probe (the experimental data are indicated by reference numerals 81c and 82c). FIG. 8A shows examples of experimental data for the treatment with 600 μm laser fiber of an Ho:YAG laser, and 4.5 Fr NPL probe, whereas FIG. 8B shows examples of experimental data for the treatment with 365 μm laser fiber of an Ho:YAG laser, and 3.6 Fr NPL probe. The same diode laser with 365 μm laser fiber was used for the experiments shown in FIGS. 8A and 8B.

The experimental data show that energy consumption, and thus the time to complete fragmentation of a concretion, drops significantly when the combined treatment is used, as compared to the use of each method separately. Furthermore, it was found that the amount of reduction of cumulative energy spent for the first breakage of the concretion is not significantly dependent on the type of the laser used for treating the concretion surface (i.e. the difference is about twice the amount). However, the amount of reduction of the cumulative energy required for the first breakage by the combined technique (i.e., NPL treatment after laser treatment) is reduced by about an order of magnitude as compared to the energy required for the first breakage when only the NPL treatment was applied. Moreover, there also occurs a substantial reduction (by up to several times) of the cumulative energy and, thus, the time it takes for the stone to disintegrate completely, which is of vital importance in clinical practice.

Therefore, the initial use of laser radiation to irradiate the calculous surface, with a further fragmentation of the calculus accomplished by using the NPL device is an efficient method that makes it possible to substantially reduce the total cumulative energy and the time required for the fragmentation of various size and dense calculi.

As such, those skilled in the art to which the present invention pertains, can appreciate that while the present invention has been described in terms of preferred embodiments, the concept upon which this disclosure is based may readily be utilized as a basis for the designing of other structures and processes for carrying out the several purposes of the present invention.

It should be understood that the medical device of the present invention is not limited to medical treatment of a human body. It can be successfully employed for medical treatments of animals as well.

Moreover, the present invention is not limited to medical procedures, and may be used to shatter and extract any type of article from a wide range of inaccessible locations, such as inside a pipe or tube (for example, the waste outlet of a domestic sink) or inside a chamber within a large piece of machinery which would be difficult to dismantle.

Also, it is to be understood that the phraseology and terminology employed herein are for the purpose of description and should not be regarded as limiting.

It is important, therefore, that the scope of the invention is not construed as being limited by the illustrative embodiments set forth herein. Other variations are possible within the scope of the present invention as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1. A medical device for breaking an organo-mineral concretion into smaller pieces, comprising:
a combined probe including a laser waveguide probe and a nanosecond electro-pulse lithotripter probe;
an optical energy source coupled to the laser waveguide probe and configured to generate a laser radiation field having energy sufficient to form a defect on a surface of the organo-mineral concretion when the laser waveguide probe is applied to the organo-mineral concretion; and
an electrical energy source coupled to the nanosecond electro-pulse lithotripter probe and configured to generate high-voltage nanosecond electro-pulses having energy sufficient to break the organo-mineral concretion by providing a spark electrical discharge through the organo-mineral concretion when the nanosecond electro-pulse lithotripter probe is applied to the organo-mineral concretion; and
a monitoring and control system configured for monitoring operation parameters and controlling operation of the device by switching operation of the device from the activating of the laser waveguide probe for generating laser radiation to the activating of the nanosecond electro-pulse lithotripter probe for generating nanosecond electric pulses.

2. The medical device of claim 1, wherein the laser waveguide probe includes at least one laser fiber for providing the laser radiation to the organo-mineral concretion, and wherein the nanosecond electro-pulse lithotripter probe includes an operating head configured to provide spark electrical discharge through the organo-mineral concretion.

3. The medical device of claim 2, wherein a distal portion of said at least one laser fiber is arranged coaxially with the operating head of the nanosecond electro-pulse lithotripter probe.

4. The medical device of claim 3, wherein said at least one laser fiber is arranged along the longitudinal axis of the combined probe, and wherein the operating head includes lithotripter electrodes formed as concentrically placed tubular bushings surrounding the laser fiber.

5. The medical device of claim 3, wherein said at least one laser fiber has a tubular shape, and the operating head is arranged within the laser fiber.

6. The medical device of claim 3, wherein the operating head of the electro-pulse lithotripter and the laser fiber are movable independently of one another.

7. The medical device of claim 1, wherein the combined probe includes an external sheath surrounding the laser waveguide probe and a nanosecond electro-pulse lithotripter probe.

8. The medical device of claim 2, wherein said at least one laser fiber of the laser waveguide probe is arranged in parallel relation to the operating head of the nanosecond electro-pulse lithotripter probe.

9. A method for breaking a organo-mineral concretion into smaller pieces, comprising:
providing the device of claim 1;
generating the laser radiation field having energy sufficient to form a defect on the surface of the organo-mineral concretion;
generating the high-voltage nanosecond electric pulses having energy sufficient to break the organo-mineral concretion by providing the spark electrical discharge through the organo-mineral concretion;
applying the laser waveguide probe to the surface of the concretion, and treating the surface with a laser radiation field to create a defect; and
applying the nanosecond electro-pulse lithotripter probe to the treated surface to provide a spark electrical discharge through the concretion.

10. The method of claim 9, wherein said applying of the nanosecond electro-pulse lithotripter probe is carried out to the treated surface after said applying the laser waveguide probe to the treated surface.

11. The method of claim 10, wherein said applying of the nanosecond electro-pulse lithotripter probe is carried out on the area of the defect created by the laser waveguide probe.

12. The method of claim 9, wherein the range of laser wavelengths is in the range of 0.94 μm-10.6 μm.

13. The method of claim 9, wherein the total cumulative energy during laser surface treatment lies within the range of a few Joules to several thousand Joules.

14. The method of claim 13, wherein the total cumulative energy during laser surface treatment lies within the range of 15 Joules to 250 Joules.

15. The method of claim 9, wherein the surface is treated by continuous laser radiation.

16. The method of claim 9, wherein the surface is treated by pulsed laser radiation.

17. The method of claim 16, wherein a duration of pulses of the pulsed laser radiation is in the range of 0.1 ms to 60 ms, with a pulse frequency in the range of 1 Hz to 30 Hz and power in the range of 0.5 W to 40 W.

18. The method of claim 12, wherein an energy in the pulse is in the range of 0.3 Joule to 5 Joules.

19. The method of claim 9, wherein an amplitude of the electro-pulses of the electro-pulse lithotripter probe is in the range of 5 kV to 20 kV, and energy in the pulse is in the range of 0.1 Joule to 2 Joules.

20. The method of claim 16, wherein a single high-voltage nanosecond electric-pulse is used.

21. The method of claim 19, wherein a train of high-voltage nanosecond electric-pulses is applied at a frequency in the range of 3 Hz to 20 Hz.

22. The method of claim 9, comprising a multiple sequential treatment of a concretion with the laser waveguide probe and a nanosecond electro-pulse lithotripter probe.
